(19) United States
(12) Patent Application Publication
(10) Pub. No.: US 2019/0088356 A1
(43) Pub. Date: Mar. 21, 2019

(54) System and Method for a Payment Exchange Based on an Enhanced Patient Care Plan

(71) Applicant: Parkland Center for Clinical Innovation, Dallas, TX (US)

(72) Inventors: George Oliver, Southlake, TX (US); Steve Miff, Dallas, TX (US); Keith Kosel, Highland Village, TX (US); Vikas Chowdhry, Southlake, TX (US); Priyanka Kharat, Dallas, TX (US)

(21) Appl. No.: 16/194,277

(22) Filed: Nov. 16, 2018

Related U.S. Application Data
(63) Continuation-in-part of application No. 14/514,164, filed on Oct. 14, 2014.
(60) Provisional application No. 61/891,054, filed on Oct. 15, 2013.

(51) Int. Cl. G16H 20/40; G16H 20/60; G16H 20/10; G16H 50/30; G16H 15/00; G06Q 40/08; G16H 10/60; G09B 19/00

(52) U.S. Cl. CPC G16H 20/40; G16H 20/60; G16H 20/10; G09B 19/0092; G16H 15/00; G06Q 40/08; G16H 10/60; G16H 50/30

(57) ABSTRACT

A patient care plan system includes a repository of patient data including real-time clinical and non-clinical data that include data generated as a result of at least one treatment received by the patient provided by an outside service provider; at least one predictive model configured to analyze clinical and social factors derived from the patient's data to determine a risk score associated with the particular medical condition; a patient care plan module configured to selectively extract data from the patient's data to generate a targeted patient data summary including data that are indicative of quality metrics associated with the at least one treatment received by the patient, and organize and format the extracted data into a patient care plan; and a payment interface module configured to transmit the patient's care plan to a payor in exchange for payment for the at least one treatment received by the patient.

### RELATED APPLICATION

This patent application is a continuation-in-part of U.S. application Ser. No. 14/514,164 filed on Oct. 14, 2014, which claims the benefit of U.S. Provisional Application No. 61/891,054 filed on Oct. 15, 2013, all of which is incorporated herein by reference. This application is also related to the following patents, all of which are incorporated herein by reference: U.S. Pat. No. 9,536,052 filed on Sep. 13, 2012, entitled “Clinical Predictive and Monitoring System and Method”; and U.S. Pat. No. 9,147,041 filed on Sep. 5, 2013, entitled “Clinical Dashboard User Interface System and Method.”

### FIELD

The present disclosure relates to a computer system, and more particularly to a system and method for a payment exchange based on a patient care plan.

### BACKGROUND

The Continuity of Care Document (CCD) is an electronic document exchange specification for sharing patient summary information between entities. The CCD is a compromise reached by two standards groups, ASTM International (American Section of the International Association for Testing Materials) and Health Level 7 (HL7). The specific content and scope of the CCD was determined by another specification, ASTM's Continuity of Care Record (CCR), an XML-based specification for patient summary data. The summary includes the commonly needed pertinent information about the patient's current and past health status in a form that can be shared by all computer applications, including web browsers, electronic medical record (EMR) and electronic health record (EHR) software systems. While some suggest that the CCD standard competes with the CCR standard, HL7 considers the CCD standard an implementation of the CCR standard. A CCD document is not intended to be a complete medical history for a given patient, but it is intended to include only a snapshot of patient information critical to effectively continue care of the patient. This snapshot of information has 17 different sections, which include the clinical content as defined originally by the CCR. Some sections, such as Family History, may include information from outside of the defined snapshot of time.

### BRIEF DESCRIPTION OF THE DRAWINGS

FIG. 1 is a simplified block diagram of an exemplary embodiment of a system and method of a payment exchange based on a patient care plan 10 according to the teachings of the present disclosure;

FIG. 2 is a simplified logical diagram of an exemplary embodiment of a predictive analytic engine 40 according to the present disclosure;

FIG. 3 is a simplified block diagram of an exemplary embodiment of a patient web portal 22 of the system and method of a payment exchange based on a patient care plan 10 according to the teachings of the present disclosure; and

FIG. 4 is a simplified flow diagram illustrating the system and method of a payment exchange based on a patient care plan 10 according to the teachings of the present disclosure.

### DETAILED DESCRIPTION

Although the system and method for a payment exchange based on a conventional or enhanced patient care plan described herein are applicable to other diseases, kidney disease is the focus of the description hereinafter as a prime example of how disease progression can be slowed down and patient benefit from implementation thereof. A diagnosis of kidney disease means that a person's kidneys are damaged and cannot effectively filter blood to remove waste and excess fluids, leading to a build-up of harmful waste in the patient's body. Major risk factors for kidney disease include diabetes, high blood pressure, and family history of kidney failure. In turn, a person with kidney disease has an increased chance of stroke and heart attack. A patient with chronic kidney disease (CKD) experiences a reduction of kidney function over a period of time that may lead to end-stage renal disease (ESRD). Dialysis is an artificial means used to treat patients with ESRD to filter and remove waste and excess fluids from the patient's blood. Treatment costs especially escalate substantially in later stage CKD and during transition to dialysis and/or transplant. A large percentage of patients with this condition are unaware of their CKD condition, and a majority of them are unaware of the importance of preventative measures to slow its progression.

CKD is a serious disease as it kills more people than breast or prostate cancer each year. CKD is present in approximately 20 percent of the general population in the United States, with more than 661,000 Americans have kidney failure. Of these, 468,000 individuals are on dialysis, and roughly 193,000 live with a functioning kidney transplant. In 2013, more than 47,000 Americans died from kidney disease. Although the number of ESRD incident cases plateaued in 2010, the number of ESRD prevalent cases continues to rise by about 21,000 cases per year. In the U.S., the cost for the treatment of CKD and ESRD is likely to exceed $48 billion per year. Treatment for ESRD consumes 6.7% of the total Medicare budget to care for less than 1% of the covered population.

The prevalence of CKD in the U.S. veteran population is estimated to be as high as 40 percent of the veteran population due to demographic differences and the existence of significant co-morbidities associated with CKD in the veteran population—diabetes mellitus and hypertension. It is estimated that an additional 5 percent of the veteran population may have undiagnosed CKD. The Veterans Affairs (VA) spends upwards of $18 Billion on the care of patients with CKD and ESRD. Although some VA facilities are equipped to perform dialysis, the majority of veterans are referred out and treated by outside dialysis providers, the services by which are then reimbursed and paid for by the VA.

Understandably, the VA wants oversight over the care of the veterans by these external service providers. The VA wants assurances that the dialysis and associated services have been satisfactorily delivered by these external service providers and that quality metrics are satisfied before payment for the services is made. For example, quality metrics may include measurable values that indicate adverse impact on the glomerular filtration rate (GFR), such as blood pressure control, Renin-Angiotensin Axis blockade, and glycemic control. More specifically, the VA wants to be able to determine and monitor quality of service, compliance, vascular access, dialysis, and transplant status/interest of the veterans receiving outside care. Also important is enabling the patient themselves to be able to easily access and understand his/her own care plan, lab values, and informational/educational materials related to CKD to help slow the progression of the disease to ESRD. Modeling data suggest that the cumulative economic impact of slowing the progression of CKD, even by as little as 10%, would be staggering. There is thus strong support for the development and implementation of intensive reno-protective efforts beginning at the early stages of chronic kidney disease and continued throughout its course. While lifetime incidence of ESRD approaches 3%, approximately 11% of persons who reach stage 3 will eventually progress to stage 5. Targeting awareness programs and fostering achieving the best preventative care in this population has the highest rates of impact on cost models developed by for the National Institute of Diabetes and Digestive and Kidney Diseases (NIDDK).

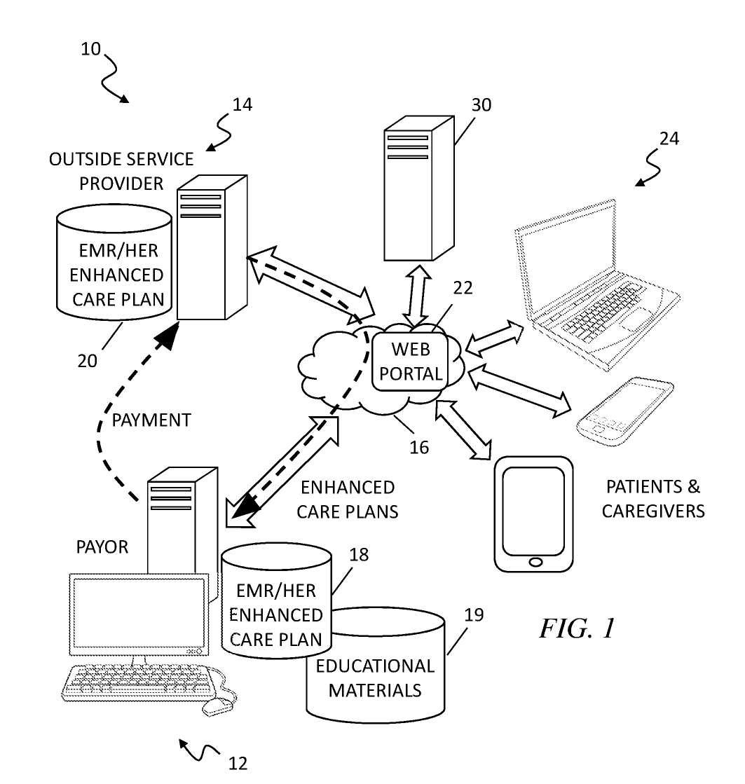

FIG. 1 is a simplified block diagram of an exemplary embodiment of a system and method for a payment exchange based on a conventional or enhanced patient care plan 10 according to the teachings of the present disclosure. In FIG. 1, reference numeral 12 is used to refer to the computer system(s), network(s), and database(s) of a payor (e.g., the VA), and reference numeral 14 is used to refer to the computer system(s), network(s), and database(s) of an outside service provider. Both systems 12 and 14 can communicate with each other via the Internet or another computer network 16. System 10 preferably employs a web application using the HTMLS standard that will use FHIR (Fast Healthcare Interoperability Resources) data interfaces to exchange a conventional or enhanced patient care plan that will be used as the basis for payment reimbursement for services rendered. Further, the patient care plan facilitates coordination of care of the patient between the VA and outside service providers. The FHIR Specification is a draft standard describing data formats and elements and an application programming interface (API) for exchanging electronic health records (EHR). The standard was created by the Health Level Seven International (HL7) health-care standards organization. The system 10 uses web services to authenticate and retrieve selected patient data (EHR/EMR). The web service content is in HL7 compliant message formats.

Following the chronic kidney disease example, the system produces an enhanced care plan that will be a CKD-specific version of the continuity of care document (CCD) with additional quality metric information. The term “enhanced care plan” is herein used to refer to a targeted digest of patient data intended to facilitate and coordinate patient care such as the CCD or other versions thereof with additional quality metrics and other information included for the primary purpose of payment for services. Quality metrics that may be included as part of the goals of care in the enhanced care plan are: chronic use of nonsteroidal anti-inflammatory drugs; metformin used below eGFR minimum; blood pressure at target goal; accelerated decline in eGFR above average rates of decline; reports of acute kidney injury in the previous year; increase in proteinuria; use of angiotensin converting enzyme inhibitors or angiotensin receptor blockers; episodes of recurrent hyperkalemia; admissions to the hospital; documented instances of anemia; absent referral to Nephrology for CKD stage 4 patients; and risk assessment of CKD progression to a higher stage in the next year. The risk assessment is made by a predictive analytic engine analyzing the patient's lab values, e.g., eGFR (estimated glomerular filtration rate), urine protein level, protein/creatinine ratio, and microalbumin/creatinine ratio, and other factors, including social and non-clinical factors.

As shown, the payor computer system 12 includes database(s) 18 and 19 to house patient EMR/HER, and conventional and enhanced patient care plans, as well as informational and educational materials (including text, charts, graphics, videos, etc.) related to CKD. Similarly, the computer system 14 of the outside service provider(s) also include a database 20 to store patient EMR/EMR, and conventional and enhanced patient care plans. A web portal 22 is further provided to enable the patients and perhaps their caregivers to access the patients' EHR/EMR, patient care plan, and informational/educational materials using a variety of computer devices 24, including mobile telephones, notebook computers, laptop computers, desktop computers, wearable computer devices, etc. The primary care practitioner (PCP) at the VA and/or outside service provider may provide recommendations on diet, exercise, and medications via the web portal 22. The patient may, through the web portal 22, provide comments, notes, and pose questions to the healthcare team at the VA and/or the outside service provider. The VA and outside provider physicians may access the patient's care plan that organizes the patient's summary clinical and non-clinical data in a easy to read and organized manner. For example, the physicians may access a problem list, lab trends, medications and refill pattern, over-the-counter supplements, diet history, and next appointments. The VA and outside provider care team may also send each other messages about the treatment and care of particular patients.

The system 10 implementation may be based on, e.g., a microservices architecture with responsive, cross platform compatible web applications at the front-end. A microservices architecture structures an application as a collection of loosely coupled services that are independently deployable.

The system and method for a payment exchange based on a patient care plan 10 includes a predictive analytic engine 40 that is configured to receive and analyze a variety of clinical and non-clinical (social services) data relating to the patients. The variety of data may include real-time data streams and historical or stored data from a plurality of data sources 30 (represented in FIG. 1 by a computer server) including, e.g., hospitals and healthcare entities, non-health care entities, health information exchanges, social-to-health information exchanges, and social services (case management) entities. The system 10 may use these data to determine a disease risk score for a particular medical condition, e.g., progression of CKD or onset of ESRD, for a patient so that he/she receives more targeted intervention, treatment, care, and informational/educational materials that are tailored and customized to the particular patient's health condition and disease.

The patient data received by the system 10 may include electronic medical records (EMR) that include both clinical and non-clinical data. The EMR clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing. Additional sources or devices of EMR data may provide, for example, lab results, medication assignments and changes, EKG results, radiology notes, daily weight readings, and daily blood sugar testing results from wearable devices.

The EMR non-clinical data may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; community and religious organizational involvement; census tract location and census reported socioeconomic data for the tract; requirements for governmental living assistance; ability to make and keep medical appointments; independence on activities of daily living; hours of seeking medical assistance; location of seeking medical services; sensory impairments; cognitive impairments; mobility impairments; employment; and economic status in absolute and relative terms to the local and national distributions of income; climate data; and health registries. The non-clinical patient data may further include data entered by the patients, such as data entered or uploaded to a patient portal.

The system 10 may further receive data from health information exchanges (HIE). HIEs are organizations that mobilize healthcare information electronically across organizations within a region, community or hospital system. HIEs are increasingly developed to share clinical and non-clinical patient data between healthcare entities within cities, states, regions, or within umbrella health systems. Data may arise from numerous sources such as hospitals, clinics, consumers, payers, physicians, labs, outpatient pharmacies, ambulatory centers, nursing homes, and state or public health agencies, and patient care facilities.

A subset of HIEs connect healthcare entities to community organizations that do not specifically provide health services, such as non-governmental charitable organizations, social service agencies, and city agencies. The system 10 may receive data from these social services organizations and social-to-health information exchanges, which may include, for example, information on daily living skills, availability of transportation to medical appointments, employment assistance, training, substance abuse rehabilitation, counseling or detoxification, rent and utilities assistance, homeless status and receipt of services, medical follow-up, mental health services, meals and nutrition, food pantry services, housing assistance, temporary shelter, home health visits, domestic violence, appointment adherence, discharge instructions, prescriptions, medication instructions, neighborhood status, and ability to track referrals and appointments.

Another source of data may include social media or social network services, such as FACEBOOK, GOOGLE+, TWITTER, and other websites can provide information such as the number of family members, relationship status, identification of individuals who may help care for a patient, and health and lifestyle preferences that may influence health outcomes. These social media data may be received from the websites, with the individual's permission, and some data may come directly from a user's computing devices (mobile phones, tablet computers, laptops, etc.) as the user enters status updates, for example.

These non-clinical or social patient data may potentially provide a much more realistic and accurate depiction of the patient's overall holistic healthcare environment. Augmented with such non-clinical patient data, the analysis and predictive modeling to identify patients at high-risk of CKD progression become much more robust and accurate.

As shown in FIG. 1, the system 10 may receive data streamed in real-time as well as from historic or batched data from various data sources 30 in a wide variety of formats according to a variety of protocols, including FHIR, CCD, XDS, HL7, SSO, HTTPS, EDI, CSV, etc. The data may be encrypted or otherwise secured in a suitable manner. The data may be pulled (polled) by the system 10 from the various data sources 30 or the data may be pushed to the system 10. Alternatively or in addition, the data may be received in batch processing according to a predetermined schedule or on-demand. The databases may include one or more local servers, memory, drives, and other suitable storage devices. Alternatively or in addition, the data may be encrypted and stored in a data center in the cloud and accessed via a global computer network.

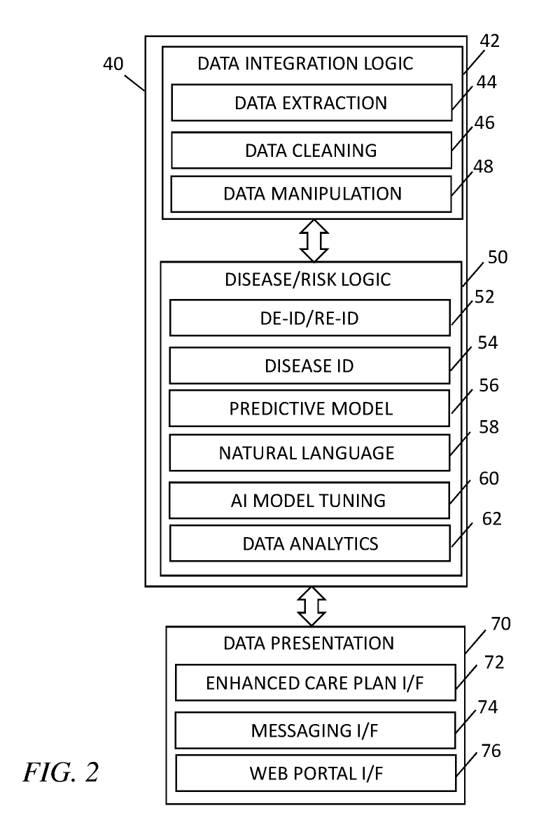

FIG. 2 is a simplified logical block diagram of an exemplary embodiment of a predictive analytic engine 40 in the system and method for a payment exchange based on a patient care plan 10 according to the teachings of the present disclosure. Because the system 10 receives and extracts data from many disparate data sources 30 in myriad formats pursuant to different protocols, the incoming data first undergo a multi-step process before they may be properly analyzed and utilized. The predictive analytic engine 40 includes a data integration logic module 42 that further includes a data extraction process 44, a data cleansing process 46, and a data manipulation process 48. It should be noted that although the data integration logic module 42 is shown to have distinct processes 44-48, these are done for illustrative purposes only and these processes may be performed in parallel, iteratively, and interactively.

The data extraction process 44 extracts clinical and non-clinical data from the plurality of data sources 30 in real-time or in historical batch files either directly or through the Internet, using various technologies and protocols. Preferably in real-time, the data cleansing process 46 “cleans” or pre-processes the data, putting structured data in a standardized format and preparing unstructured text for natural language processing (NLP) to be performed in the disease/risk logic module 50 described below. The system may also receive “clean” data and convert them into desired formats (e.g., text date field converted to numeric for calculation purposes).

The data manipulation process 48 may analyze the representation of a particular data feed against a meta-data dictionary and determine if a particular data feed should be re-configured or replaced by alternative data feeds. For example, a given hospital EMR may store the concept of “maximum creatinine” in different ways. The data manipulation process 48 may make inferences in order to determine which particular data feed from the EMR would best represent the concept of “creatinine” as defined in the meta-data dictionary and whether a feed would need particular re-configuration to arrive at the maximum value (e.g., select highest value).

The data integration logic module 42 then passes the pre-processed data to a disease/risk logic module 50, which is operable to calculate a risk score associated with an identified disease or condition for each patient and to identify those patients who should receive targeted intervention and care. The disease/risk logic module 50 includes a de-identification/re-identification process 52 that is operable to remove all protected health information according to HIPAA standards before the data is transmitted over the Internet. It is also adapted to re-identify the data. Protected health information that may be removed and added back may include, for example, name, phone number, facsimile number, email address, social security number, medical record number, health plan beneficiary number, account number, certificate or license number, vehicle number, device number, URL, all geographical subdivisions smaller than a state, including street address, city, county, precinct, zip code, and their equivalent geocodes (except for the initial three digits of a zip code, if according to the current publicly available data from the Census Bureau), Internet Protocol number, biometric data, and any other unique identifying number, characteristic, or code.

The disease/risk logic module 50 further includes a disease identification process 54 that is adapted to identify one or more diseases or conditions of interest for each patient. The disease identification process 54 considers data such as lab orders, lab values, clinical text and narrative notes, and other clinical and historical information to determine the probability that a patient has a particular disease. Additionally, during disease identification, natural language processing is conducted on unstructured clinical and non-clinical data to determine the disease or diseases that the physician believes are prevalent. This process 54 may be performed iteratively over the course of many days to establish a higher confidence in the disease identification as the physician becomes more confident in the diagnosis. New or updated patient data may not support a previously identified disease, and the system would automatically remove the patient from that disease list. The natural language processing combines a rule-based model and a statistically-based learning model.

The disease identification process 54 utilizes a hybrid model of natural language processing, which combines a rule-based model and a statistically-based learning model. During natural language processing, raw unstructured data, for example, physicians' notes and reports, first go through a process called tokenization. The tokenization process divides the text into basic units of information in the form of single words or short phrases by using defined separators such as punctuation marks, spaces, or capitalizations. Using the rule-based model, these basic units of information are identified in a meta-data dictionary and assessed according to predefined rules that determine meaning. Using the statistical-based learning model, the disease identification process 54 quantifies the relationship and frequency of word and phrase patterns and then processes them using statistical algorithms. Using machine learning, the statistical-based learning model develops inferences based on repeated patterns and relationships. The disease identification process 54 performs a number of complex natural language processing functions including text pre-processing, lexical analysis, syntactic parsing, semantic analysis, handling multi-word expression, word sense disambiguation, and other functions.

The disease/risk logic module 50 further comprises a predictive model process 56 that is adapted to predict the risk of particular disease, condition, or adverse clinical and non-clinical event of interest according to one or more predictive models. For example, if the hospital desires to determine the level of risk for future readmission for all patients currently admitted with heart failure, the heart failure predictive model may be selected for processing patient data. However, if the hospital desires to determine the risk levels for all internal medicine patients for any cause, an all-cause readmissions predictive model may be used to process the patient data. As another example, if the VA desires to identify those patients most at risk for short-term and long-term diabetic complications, the diabetes predictive model may be used to target those patients. Sticking with the CKD example, the VA may use the predictive model to identify the top 2% of its patient population that are at risk of developing ESRD. Other predictive models may include HIV readmission, diabetes identification, risk for cardio-pulmonary arrest, acute coronary syndrome, pneumonia, cirrhosis, all-cause disease-independent readmission, colon cancer pathway adherence, risk of hunger, loss of housing, and others.

Continuing to use the prior example, the predictive model for CKD progression may take into account a set of risk factors or variables, including the values for laboratory and vital sign variables such as: serum creatinine, creatinine clearance, glomerular filtration rate, urine albumin, urine microalbumin, albumin to creatinine ratio, blood urea nitrogen, diastolic blood pressure, systolic blood pressure, etc. Further, non-clinical factors may also be considered, for example, dietary considerations, risky health behaviors (e.g., use of illicit drugs or substance), number of emergency room visits in the prior year, past adherence to doctor appointments, past compliance of medication regimen, and other factors. The predictive model specifies how to categorize and weight each variable or risk factor, and the method of calculating the predicted probability of disease progression. In this manner, the predictive analytic engine 40 is able to rank and stratify, in real-time, the risk of each patient in developing CKD and ESRD. Therefore, those patients at the highest risks are automatically identified so that targeted intervention and care may be instituted. One output from the disease/risk logic module 50 includes the risk scores of all the patients for a particular disease or condition. The module 50 may rank or stratify the patients according to their respective risk scores, and provide a list of those patients at the top of the list who are most at risk for targeted intervention. For example, the VA may desire to identify the top X number of patients who are most at risk for renal failure, and/or the top 5% of patients most at risk for CKD progression. Other diseases and conditions that may also be identified using predictive modeling, including, e.g., congestive heart failure and diabetes.

The disease/risk logic module 50 may further include a natural language generation module 58, which is adapted to receive the output from the predictive model 56 such as the risk score and risk variables for a patient, and “translate” the data to present, in the form of natural language, the evidence that the patient is at high-risk for that disease or condition. This module thus provides the intervention coordination team with additional information that supports why the patient has been identified as high-risk for the particular disease or condition. In this manner, the intervention coordination team may better formulate the targeted inpatient and outpatient intervention and treatment plan to address the patient's specific situation.

The natural language generation module 58 also provides summary information about a patient, such as demographic information, medical history, primary reason for the visit, etc. This summary statement provides a quick snapshot of relevant information about the patient in narrative form.

The disease/risk logic module 50 may further include an artificial intelligence (AI) model tuning process 60, which utilizes adaptive self-learning capabilities using machine learning technologies. The capacity for self-reconfiguration enables the system 10 to be sufficiently flexible and adaptable to detect and incorporate trends or differences in the underlying patient data or population that may affect the predictive accuracy of a given algorithm. The artificial intelligence model tuning process 60 may periodically retrain a selected predictive model for improved accurate outcome to allow for selection of the most accurate statistical methodology, variable count, variable selection, interaction terms, weights, and intercept for a local health system or clinic. The artificial intelligence model tuning process 60 may automatically modify or improve a predictive model in three exemplary ways. First, it may adjust the predictive weights of clinical and non-clinical variables without human supervision. Second, it may adjust the threshold values of specific variables without human supervision. Third, the artificial intelligence model tuning process 60 may, without human supervision, evaluate new variables present in the data feed but not used in the predictive model, which may result in improved accuracy. The artificial intelligence model tuning process 60 may compare the actual observed outcome of the event to the predicted outcome then separately analyze the variables within the model that contributed to the incorrect outcome. It may then re-weigh the variables that contributed to this incorrect outcome, so that in the next reiteration those variables are less likely to contribute to a false prediction. In this manner, the artificial intelligence model tuning process 60 is adapted to reconfigure or adjust the predictive model based on the specific clinical setting or population in which it is applied. Further, no manual reconfiguration or modification of the predictive model is necessary. The artificial intelligence model tuning process 60 may also be useful to scale the predictive model to different health systems, populations, and geographical areas in a rapid timeframe.

As an example of how the artificial intelligence model tuning process 60 functions, the sodium variable coefficients may be periodically reassessed to determine or recognize that the relative weight of an abnormal sodium laboratory result on a new population should be changed from 0.1 to 0.12. Over time, the artificial intelligence model tuning process 60 examines whether thresholds for sodium should be updated. It may determine that in order for the threshold level for an abnormal sodium laboratory result to be predictive for readmission, it should be changed from, for example, 140 to 136 mg/dL. Finally, the artificial intelligence model tuning process 60 is adapted to examine whether the predictor set (the list of variables and variable interactions) should be updated to reflect a change in patient population and clinical practice. For example, the sodium variable may be replaced by the NT-por-BNP protein variable, which was not previously considered by the predictive model.

The disease/risk logic module 50 may further include a data analytics module 62 that analyzes the data processed by the disease/risk logic module 50 and performs certain data processing procedures related to the presentation of the data. The data analytics module 62 performs tasks such as identifying data that are relevant to the information to be displayed by a widget, analyze patient input to identify medical terms or jargon for which the patient is seeking information, and identify relevant resources to recommend to the patient.

The results from the disease/risk logic module 50 are provided to the hospital personnel, such as the intervention coordination team, other caretakers, and the patient, by a data presentation logic module 70. The data presentation logic module 70 includes a patient care plan interface 72 that is configured to provide organized presentation of the targeted patient data summary to the patient and VA/clinical personnel.

The data presentation logic module 70 further includes a messaging interface 74 that is adapted to generate output messages in forms such as HL7 messaging, text messaging, e-mail messaging, multimedia messaging, REST, XML, computer generated speech, constructed document forms containing graphical, numeric, and text summary of the risk assessment, reminders, and recommended actions. The interventions generated or recommended by the system 10 may include: patient care plan; risk score report to the primary care physician to highlight risk of kidney disease progression for certain patients; comparison of aggregate risk of kidney disease progression for a single outside service provider or risk-standardized comparisons of the rates of kidney disease progression among outside service providers; and HL7 message to communicate kidney disease progression risk of certain patients to VA physicians. The information provided would highlight potential strategies to slow kidney disease progression including: recommendations for diet, exercise, and medications.

This output may be transmitted wirelessly or via LAN, WAN, the Internet, and delivered to the VA facilities' electronic medical record stores, user electronic devices (e.g., pager, text messaging program, mobile telephone, tablet computer, mobile computer, laptop computer, desktop computer, and server), health information exchanges, and other data stores, databases, devices, and users. At the VA, the patient care plans may be automatically formatted for printing, analyzed for reporting (e.g., identify the top 1% of patients who are at risk of CKD progression), and analyzed to determine the outstanding payment amount for services rendered by the outside service providers, etc. The analysis may determine that, according to the quality metrics, a certain outside service provider is not entitled to the full outstanding amount and instead a certain percentage pursuant to a value-based or alternative payment model. The patient care plan allows documentation of services rendered and quality metrics, and further allows additional quality checks against the possibility of billing discrepancies.

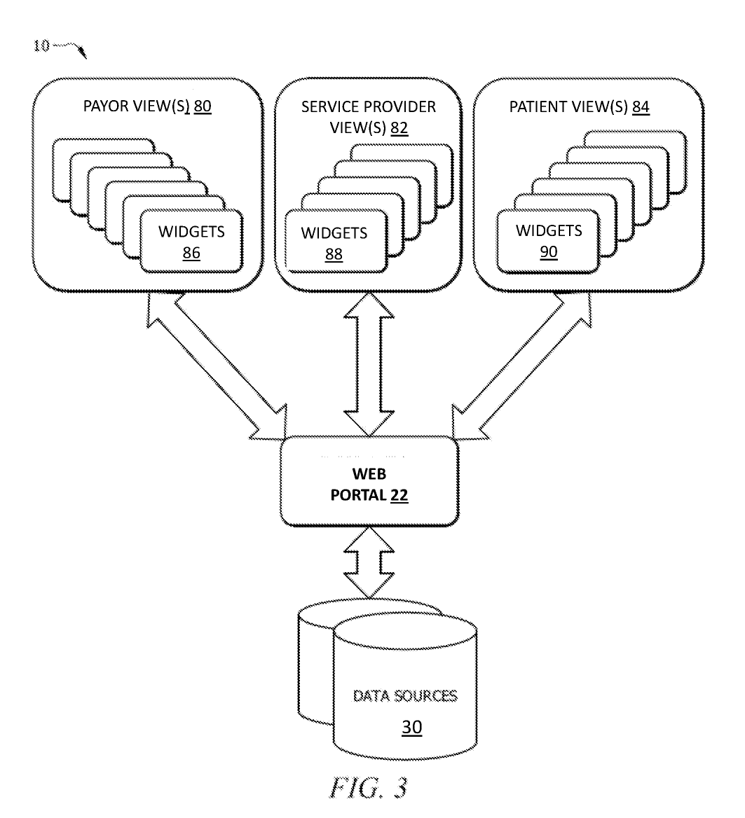

The data presentation and system configuration logic module 70 further includes a web portal 76 that is operable to present information in text, graphical, pictorial, video, and other formats accessible by web browser applications executing on a variety of computing platforms. Additional details of the operations of the web portal 22 are described below with reference to FIG. 3.

The system 10 is adapted to provide a real-time electronic summary or view of a patient's medical information in the form of the patient care plan. In a preferred embodiment, the system 10 uses predictive models, natural language processing, artificial intelligence, and other sophisticated algorithms and analytics tools to processes patient clinical and non-clinical data to identify those patients who are most at risk for developing a certain medical condition such as CKD. The quality metrics included in the patient care plan are used as the basis for value-based compensation for services rendered.

Referring to FIG. 3, the exemplary system 10 is operable to present real-time data and information from a plurality of data sources 30 (described above and shown in FIG. 1) via a web portal 22 accessible by web browser applications. The information is presented in a number of “views” 80-84 that are focused summaries of selected relevant and critical information to specific subsets of users: VA personnel, external service provider personnel, and patients. These views 80-84 are accessible via a number of interface computing devices 12, 14, and 24 (FIG. 1) wherever and whenever data is needed. Each view 80-84 comprises one or more widgets 86-90 organized on the screen or on a page that extract, collect, and present organized focused or filtered sets of information ranging from medical conditions, demographic information, healthcare regimen, allergies, and appointment information to social services referral information. The widgets 86-90 provide organized sets of information on various topics that are displayed for viewing by VA physicians, nurses, administrators, etc. (payor view(s) 80), by physicians, nurses, and other employees of external service providers (service provider view(s) 82), and/or by patient and authorized family members (patient view(s) 84).

The following is a brief description of selected exemplary widgets and the type of information that is provided by each widget.

Patient Enhanced Care Plan Widget—Provides a summary of the patient's medical history. Through natural language processing and generation, the system 10 configures and displays a succinct text summary of the patient's relevant medical data generated by the clinical predictive analytic engine. This widget is preferably defined to be accessible from the payor and service provider views.

Predictive Analytics Widget—Provides an identification of a patient's risk for kidney disease progression. The system 10 aggregates and analyzes available patient clinical and social factors, and uses advanced algorithms to calculate a patient's risk for kidney disease progression, which can then be displayed to facilitate delivery of targeted interventions. This widget is preferably defined to be accessible from the payor and service provider views.

Allergies Widget—Provides a patient's allergies displayed with reaction symptoms and severity to help detect and prevent allergic reactions. The allergy information is extracted from the patient's Electronic Medical Record (EMR) as well as from clues found in unstructured text such as physician's notes or patient input/comments. This widget is preferably defined to be accessible from payor, service provider, and patient views.

Chart Check Issues Widget—During patient care transitions, clinical events that should be tracked or monitored may sometimes be missed by the receiving care team. By analyzing physician notes, action items or follow-up labs can be visually flagged and displayed for the receiving care team during patient care transition. This widget is preferably defined to be accessible from the payor and service provider views.

Demographic Information Widget—A patient's demographic information helps inform decisions, and is often used when assessing eligibility and enrolling individuals for services. The demographic information is extracted from the patient's Electronic Medical Record (EMR) as well as from clues found in unstructured text such as physician's notes or patient input/comments. This widget is preferably defined to be accessible from the payor, service provider, and patient views.

Documents On File Widget—Provides access to a list of stored documents that are often used for assessing eligibility and enrolling individuals for services. This view enables access to images of documents that are available from source systems across collaborating organizations. This widget is preferably defined to be accessible from the payor, service provider, and patient views.

Height and Weight Widget—Provides records of height and weight that enable the patient care team to track and flag significant fluctuations and take action if necessary. The height and weight information are typically not available for social service settings, thus their availability may provide the case worker additional insights on how to better take care of the patient. This widget is preferably defined to be accessible from the payor, service provider, and patient views.

Upcoming Appointments Widget—Provides information on the patient's upcoming appointments with the external service provider which may be helpful to inform what other needs an individual may have, and whether they are getting the necessary services to meet those needs. This widget is preferably defined to be accessible from the payor, service provider, and patient views.

Medication Reconciliation Widget—Provides information about medications to help the patient adhere to the medication regimen and help providers make clinical decisions. This widget may provide information such as names of current and discontinued medications, medication possession ratio (the percentage of time the patient has had access to the medication), cost, flagged for review due to a recent change in the patient's status, image of the medication, and patient education materials. This information is populated by the system 10 using new analytics and data extraction methods. This widget is preferably defined to be accessible from the payor, service provider, and patient views.

Most Prominent Problems Widget—Provides a list of the most prominent (e.g., severe, urgent, chronic, most relevant) medical issues or conditions for the patient. This widget eliminates the problem of redundancies and irrelevant information that most EMR records have. This information is extracted from structured and unstructured data fields in the EMR. This widget is preferably defined to be accessible from the payor, service provider, and patient views.

Complete Problem List Widget—Provides a complete list of the patient's medical issues without redundancies and irrelevant information. This information is extracted from structured and unstructured data fields in the EMR. This widget is preferably defined to be accessible from the payor, service provider, and patient views.

Relevant Historic Abnormal Results Widget—Provides any relevant historic abnormal lab results that would be helpful to inform clinical decisions. The algorithms may adapt to criteria including but not limited to: a defined time period, outside of a range that is typical for other patients with similar medical history and similar settings, association with certain disease conditions, and the patient's medical history. The system 10 also augments the algorithms by using clues found in unstructured text. This widget is preferably defined to be accessible from the payor and service provider views.

Relevant Recent Abnormal Results Widget—Provides any relevant recent abnormal lab results that would be helpful to inform clinical decisions. The algorithms may adapt to criteria including but not limited to: a defined time period, outside of a range that is typical for other patients with similar medical history and similar settings, association with certain disease conditions, and the patient's medical history. The system 10 also augments the algorithms by using clues found in unstructured text. This widget is preferably defined to be accessible from the payor and service provider views.

Relevant Unresolved Orders and Labs Widget—Provides reminders to complete any unresolved orders and labs. The algorithms may adapt to criteria including but not limited to: a defined time period, outside of a range that is typical for other patients with similar medical history and similar settings, association with certain disease conditions, and the patient's medical history. The system 10 also augments the algorithms by using clues found in unstructured text. This widget is preferably defined to be accessible from the payor and service provider views.

Current Health Issues Widget—Provides the patient with information on health issues currently experienced by the patient. The system 10 populates this information for display from the EMR and clues found in unstructured text. This widget is preferably defined to be accessible from the payor, service provider, and patient views.

Preventive Health Widget—Provides the patient with information on preventive health diets and activities. The system 10 populates this information for display from the EMR and clues found in unstructured text. This widget is preferably defined to be accessible from the payor, service provider, and patient views.

Recent Test Results Widget—Provides information to the patient about his/her recent lab results. The system 10 populates this information for display from the EMR and clues found in unstructured text. This widget is preferably defined to be accessible from the payor, service provider, and patient views.

Diabetes Complications Widget—Provides information about the patient's diabetes complications to help inform clinical decisions. The system 10 populates this information for display from the EMR and clues found in unstructured text. This widget is preferably defined to be accessible from the payor, service provider, and patient views.

Previous BP Records Widget—Provides the patient's blood pressure records to help inform clinical decisions. The system 10 populates this information for display from the EMR and clues found in unstructured text. This widget is preferably defined to be accessible from the payor, service provider, and patient views.

Processing and Translating Clinical Notes Widget—Provides a simplified version of clinical or physician notes to help the patient understand information from medical encounters. In other words, medical jargon, abbreviations, and phrases are translated to layman terms to facilitate understanding. The system also detects and corrects inconsistencies and errors. The patient care and management system 11 uses natural language processing to extract and display a simplified summary of the patient's clinical notes. This widget is preferably defined to be accessible from the clinical and patient views.

Tailored Patient Engagement Incentives Widget—Provides patient care plans that have been tailored to the specific patient to help the patient adhere to healthy behaviors and track progress toward goals. Prescriptive analytics considers the patient's medical and social data, including but not limited to missed appointments, medication adherence, functional status, social support, and comorbidities to generate recommendations and goals for a tailored patient care plan. As milestone goals are achieved (e.g., exercise and nutrition goals), patients may receive incentives (e.g. unlock new features, earn points to redeem health education materials, health apps, or health devices). This widget is preferably defined to be accessible from the patient view.

Patient Care Preferences Widget—Provides patient care plans that factor in the patient's preferences, such as location, religious practices, cultural beliefs, preferred rounding time, end of life care, etc. The patient can record their care preferences in a patient interface or view. Care providers can view these preferences in devising the patient care plan. This widget is preferably defined to be accessible from the payor, service provider, and patient views.

Integration with Patient Devices Widget—Patients who are using mobile health monitoring devices and apps. to measure and track certain vitals data, physical or activity information, nutritional intake, and other activities (e.g., blood pressure monitoring, blood sugar monitoring, heart rate, body temperature, number of steps taken, etc.) can permit the integration of these devices with the system 10. The analytic logic of the system 10 may further utilize this information to calculate risk scores for certain diseases or adverse events, for example. This widget is preferably defined to be accessible from the payor, service provider, and patient views.

Patient Assessments Widget—Using this view and interface, a patient may view, correct, and enter an assessment of their own medical history, social history, behaviors, and family history for review and discussion during an encounter with a healthcare provider or social service provider. Predictive analysis can be used to prepare initial assessments for review by the patient, to recommend questions for discussion during an encounter, and to identify informational/educational materials based on the assessment results. This widget is preferably defined to be accessible from the payor, service provider, and patient views.

Patient Calendar Widget—The patient can use this view and interface to keep track of and adhere to appointments, self-management activities, medication regimen, medication refills, and healthy behaviors. This widget is preferably defined to be accessible from the payor, service provider, and patient views.

Tailored Patient Education Modules Widget—Patient education materials and resources are selected and tailored according to the patient's health conditions and to information such as questions, concerns, or assessment results that a patient has entered. Patient education materials can help patients to better understand and manage their medical conditions and slow the progression of diseases. This widget is preferably defined to be accessible from the payor, service provider, and patient views.

Vitals Widget—Clinical users and the patient can view a patient's relevant vital measurements in a simple summary view (e.g., current and previous blood pressure and heart rate measurements). This widget is preferably defined to be accessible from the payor, service provider, and patient views.

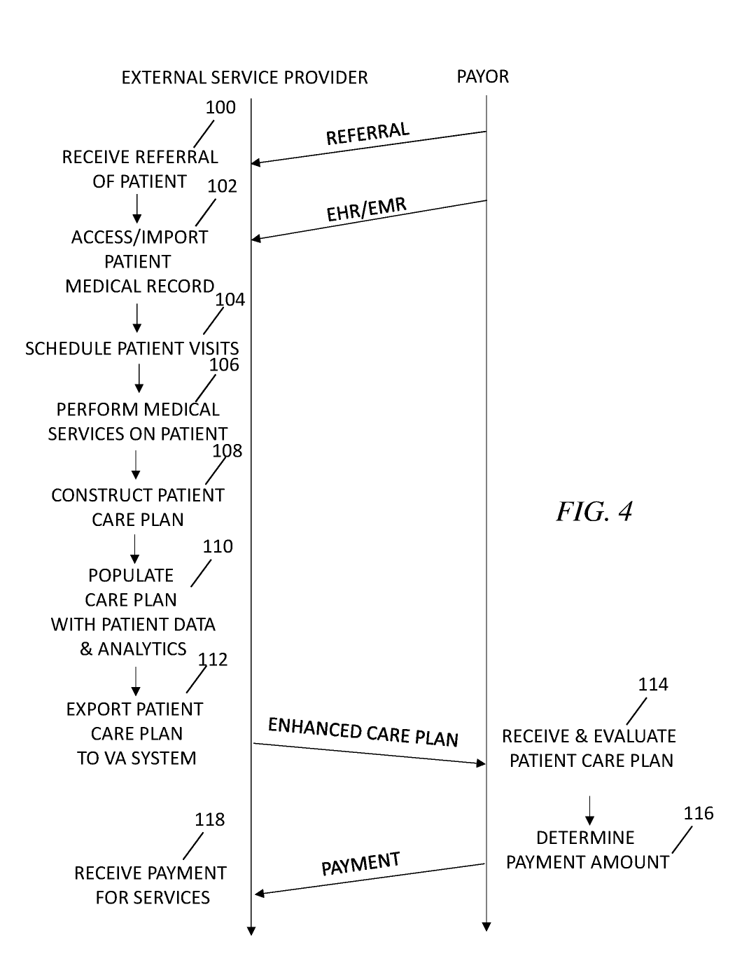

FIG. 4 is a flow diagram of an exemplary method for a payment exchange based on a patient care plan according to the teachings of the present disclosure. An external service provider, such as one that provides dialysis services, receives an electronic patient referral from a payor, such as the VA (100), for, e.g., dialysis treatment. The patient's EHR/EMR is then pushed or accessed by the external service provider, with the proper permissions and authorizations from the patient and the VA, and stored in its database (102). The referral may already include a scheduling of the patient's next visit or the external service provider schedules the patient for his/her next appointment(s) (104). The patient receives the scheduled medical services (106), and the external service provider's computer system constructs, from the patient's EHR/EMR and data associated with the patient's visits, a patient care plan (108-110). The enhanced care plan includes the patient's medical summary (including, e.g., lab data, outcome data, medication list, social work, nutrition summary, and the nephrologist's progress notes) and results from the predictive analysis of the patient's clinical and non-clinical data. This patient care plan is then exported or uploaded to the payor's computer system and stored it its database (112). The payor computer system evaluates the patient care plan to determine whether its quality metrics have been met by the services provided to the patient, and determine a payment amount based on the analysis (114-116). The service provider then receives payment for the services it rendered to the patient (118).

Although the description herein refers specifically to chronic kidney disease and providing dialysis to ESRD patients, the present system and method are applicable to any situation where a service is referred out by a payor who requires oversight and assurances that the services rendered by the outside service provider meet and are compliant with its quality metrics before payment is made to the service provider. For example, the VA may also refer its veterans to outside primary care physicians, laboratories, outpatient nephrology renal replacement therapy facilities. The timely exchange of the patient care plan enables informed collaboration among these service providers to ensure quality patient care and treatment and efficient payment for services rendered. Further, better oversight and transparency in how the ESRD patient population is serviced by outside service providers help to mitigate and prevent fraud, waste, and abuse across the VA system and improve the efficiency and integrity of the VA's payment system/process.

In addition to the use of the patient care plan to achieve oversight and transparency of the payment process, the system further helps to identify those patients at the greatest risk for CKD progression using predictive analytics, and provide patient informational/educational materials that are tailored to each patient's medical condition. The early initiation of preventative strategies to reduce the onset of novel coronary artery disease (CAD) and cerebrovascular accidents (CVA) represent the best opportunity to decrease costs as progression of CKD to CKD with coexisting congestive heart failure (CHF) increases health care cost by almost 100%. Consistent educational messaging is important to long-term management of diabetes glycemic control, reduction in cardiovascular events, immunization, blood pressure control, and statin use.

The features of the present invention which are believed to be novel are set forth below with particularity in the appended claims. However, modifications, variations, and changes to the exemplary embodiments described above will be apparent to those skilled in the art, and the patient care plan system and method described herein thus encompasses such modifications, variations, and changes and are not limited to the specific embodiments described herein.

What is claimed is:

1. A patient care plan system, comprising:
a repository of patient data including real-time clinical and non-clinical data associated with a patient, the data including data generated as a result of at least one treatment received by the patient provided by an outside service provider;
a database storing informational and educational audio/visual content associated with a particular medical condition related to the at least one treatment;
at least one predictive model configured to analyze clinical and social factors derived from the patient's clinical and non-clinical data to determine a risk score associated with the particular medical condition;
a patient care plan module configured to selectively extract data from the patient's clinical and non-clinical data to generate a targeted patient data summary including data that are indicative of quality metrics associated with the at least one treatment received by the patient, and organize and format the extracted data into an enhanced care plan configured for electronic transmission and presentation;
a web portal accessible by the patient to view the patient care plan and informational and educational audio/visual content selectively extracted from the database in response to the patient's risk score and enhanced care plan; and
a payment interface module configured to transmit the patient's care plan to a payor in exchange for payment for the at least one treatment received by the patient, the payment amount made by the payor being in response to the quality of metrics data in the patient care plan.

2. The system of claim 1, wherein the at least one predictive model is configured to analyze clinical and social factors derived from the patient's clinical and non-clinical data to determine a risk score for the progression of chronic kidney disease.

3. The system of claim 2, wherein the at least one predictive model is configured to analyze clinical and social factors derived from a plurality of patients' clinical and non-clinical data to determine a risk score for the progression of chronic kidney disease for each patient, and to stratify the plurality of patients according to their respective risk scores.

4. The system of claim 2, wherein the web portal is further configured to present a customizable patient view to provide informational and educational audio/visual content related to chronic kidney disease.

5. A patient care plan method, comprising:
receiving and storing real-time patient data including clinical and non-clinical information associated with at least one patient from at least one data source;
receiving and storing additional patient data generated in response to at least one treatment for a particular medical condition received by the patient and provided by an outside service provider;
analyzing the patient data using at least one predictive model to determine a risk score for the patient associated with the particular medical condition;
extracting data from the patient data to generate an enhanced care plan including a targeted patient data summary, risk score, and quality metrics associated with the at least one treatment received by the patient;
electronically transmitting the enhanced care plan to a payor;
receiving, at the outside service provider, a payment from the payor for the at least one treatment received by the patient in response to the patient care plan; and
providing and presenting a web portal in response to a request by the patient to view the patient care plan and informational and educational audio/visual content selectively extracted in response to the patient's risk score and patient care plan.

6. The method of claim 5, further comprising providing and presenting the patient care plan and risk score in response to a request from a healthcare provider.

7. The method of claim 5, wherein providing and presenting a web portal comprises providing and presenting a customizable patient view to provide informational and educational audio/visual content related to chronic kidney disease.

8. The method of claim 5, wherein analyzing the patient data comprises analyzing the patient data using the at least one predictive model to determine a risk score for the progression of chronic kidney disease.

9. The method of claim 5, wherein analyzing the patient data comprises analyzing data associated with a plurality of patients using the at least one predictive model to determine a risk score for each patient for the progression of chronic kidney disease.

10. The method of claim 5, further comprising evaluating, at the payor, the quality metrics and determining a payment amount for the at least one treatment received by the patient.

11. A patient care plan method, comprising:
at an outside service provider:
receiving a referral of a patient for a treatment associated with a particular medical condition from a payor;
receiving and storing real-time patient data associated with the patient including clinical and non-clinical information from the payor;
receiving and storing additional patient data generated in response to at least one treatment for the particular medical condition received by the patient;
analyzing the patient data using at least one predictive model to determine a risk score for the patient associated with the particular medical condition;
extracting data from the patient data to generate a patient care plan including a targeted patient data summary, risk score, and quality metrics associated with the at least one treatment received by the patient;
electronically transmitting the patient care plan to the payor;
at the payor:
receiving the patient care plan;
evaluating the quality metrics of the received patient care plan; and
determining a payment amount for the at least one treatment received by the patient in response to the evaluation.

12. The method of claim 11, further comprising providing and presenting the patient care plan and risk score to a web browser application in response to a request from a healthcare provider.

13. The method of claim 11, further comprising providing and presenting a web portal in response to a request by the patient to view the patient care plan and informational and educational audio/visual content selectively extracted in response to the patient's risk score and patient care plan.

14. The method of claim 13, further comprising providing and presenting a web portal comprises providing and presenting a customizable patient view to provide informational and educational audio/visual content related to chronic kidney disease.

15. The method of claim 11, wherein analyzing the patient data comprises analyzing the patient data using the at least one predictive model to determine a risk score for the progression of chronic kidney disease.

16. The method of claim 11, wherein analyzing the patient data comprises analyzing data associated with a plurality of patients using the at least one predictive model to determine a risk score for each patient for the progression of chronic kidney disease.

17. The method of claim 11, wherein the payor receives a plurality of patient care plans associated with a plurality of patients who have received at least one treatment from the outside service provider, and further comprising the payor evaluating the quality metrics in the plurality of patient care plans and determining a payment amount for the at least one treatment received by the plurality of patients.
